(19) United States
(12) Patent Application Publication
(10) Pub. No.: US 2023/0420959 A1
(43) Pub. Date: Dec. 28, 2023

(54) IMPLANTABLE BATTERY DISCONNECTION

(71) Applicant: Cochlear Limited, Macquarie University, NSW (AU)

(72) Inventors: Padraig Hurley, Frenches Forest, NSW (AU); Thomas Cooney, Redfern, NSW (AU)

(21) Appl. No.: 18/252,826

(22) PCT Filed: Oct. 14, 2021
(86) PCT No.: PCT/IB2021/059455

§ 371 Date: May 12, 2023

Related U.S. Application Data
(60) Provisional application No. 63/115,173, filed on Nov. 18, 2020.

(51) Int. Cl. H02J 7/00

(52) U.S. Cl. CPC H02J 7/0031; H02J 7/005; H02J 2310/23

(57) ABSTRACT

Presented herein are techniques for selectively disconnecting an implantable rechargeable battery of an implantable component configured to be implanted in a recipient. The implantable rechargeable battery can be temporarily disconnected to extend the calendar life of the implantable rechargeable battery and/or the implantable rechargeable battery can be permanently disconnected. While the implantable rechargeable battery is disconnected (either temporarily or permanently), the implantable component is configured to continue operation using only power signals (power) received from an external device.

### BACKGROUND

### Field of the Invention

The present invention relates generally to implantable batteries.

### Related Art

Medical devices have provided a wide range of therapeutic benefits to recipients over recent decades. Medical devices can include internal or implantable components/devices, external or wearable components/devices, or combinations thereof (e.g., a device having an external device communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: monitoring a health of at least one implantable rechargeable battery of an implantable component configured to be implanted in a recipient, wherein the at least one implantable rechargeable battery is configured to selectively power electronics of the implantable component; electrically disconnecting the at least one implantable battery such that the at least one implantable battery can no longer be charged; and while the at least one implantable rechargeable battery is disconnected, powering the electronics of the implantable component using power signals received from an external device.

In another aspect, an implantable component configured to be implanted in a recipient is provided. The implantable component comprises: an inductive coil configured to receive power signals from an external device; at least one implantable rechargeable battery configured to be charged using power signals received at the inductive coil from the external device; implant electronics configured to be powered by power received from the at least one implantable rechargeable battery or the power signals received at the inductive coil from the external device; and a battery disconnection module configured to selectively disconnect the at least one implantable rechargeable battery from the implant electronics and the inductive coil, wherein when the at least one implantable rechargeable battery is disconnected, the implant electronics are only powered using power signals received from the external device.

In another aspect, a method is provided. The method comprises: generating battery status data associated with at least one implantable rechargeable battery of an implantable component configured to be implanted in a recipient; sending the battery status data to an external computing device; receiving one or more control signals from the external computing device indicating that least one implantable rechargeable battery should be disconnected; and in response to receipt of the one or more control signals, electrically disconnecting the at least one implantable rechargeable battery such that the at least one implantable rechargeable battery can no longer be charged.

In another aspect, one or more non-transitory computer readable storage media comprising instructions are provided. The non-transitory computer readable storage media are encoded with instructions that, when executed by at least one processor, are operable to: monitor at least one implantable rechargeable battery of an implantable component configured to be implanted in a recipient, wherein the at least one implantable rechargeable battery is configured to selectively power electronics of the implantable component; and based on monitoring at least one implantable rechargeable battery, selectively electrically disconnect the at least one implantable battery such that the least one implantable battery cannot be charged.

### BRIEF DESCRIPTION OF THE DRAWINGS

Embodiments of the present invention are described herein in conjunction with the accompanying drawings, in which:

FIG. 1A illustrates a cochlear implant system, in accordance with certain embodiments presented herein;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2A is a functional block diagram of an implantable component, in accordance with certain embodiments presented herein;

FIG. 2B is a functional block diagram of another implantable component, in accordance with certain embodiments presented herein;

FIG. 3 is a flowchart of an example method, in accordance with certain embodiments presented herein;

FIG. 4 is a schematic diagram illustrating a vestibular nerve stimulator system, in accordance with certain embodiments presented herein; and

FIG. 5 is a block diagram of an external device operating with an implantable component, in accordance with certain embodiments presented herein.

### DETAILED DESCRIPTION

Presented herein are techniques for selectively disconnecting an implantable rechargeable battery of an implantable component configured to be implanted in a recipient. The implantable rechargeable battery can be temporarily disconnected to extend the calendar life of the implantable rechargeable battery and/or the implantable rechargeable battery can be permanently disconnected. While the implantable rechargeable battery is disconnected (either temporarily or permanently), the implantable component is configured to continue operation using only power signals (power) received from an external device.

As used herein, and as described further below, an implantable rechargeable battery is “connected” when the implantable rechargeable battery is capable of being charged by power signals received from an external device and is able to supply power to other operational components of the implantable component. In contrast, an implantable rechargeable battery is “disconnected” when the implantable rechargeable battery is incapable of being charged by power signals received from an external device and, in certain embodiments, is incapable of supplying power to other operational components of the implantable component.

Merely for ease of description, the techniques presented herein are primarily described with reference to a specific implantable medical device system, namely a cochlear implant system. However, it is to be appreciated that the techniques presented herein may also be implemented by other types of implantable medical devices. For example, the techniques presented herein may be implemented by other auditory prosthesis systems that include one or more other types of auditory prostheses, such as middle ear auditory prostheses, bone conduction devices, direct acoustic stimulators, electro-acoustic prostheses, auditory brain stimulators, etc. The techniques presented herein may also be used with tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

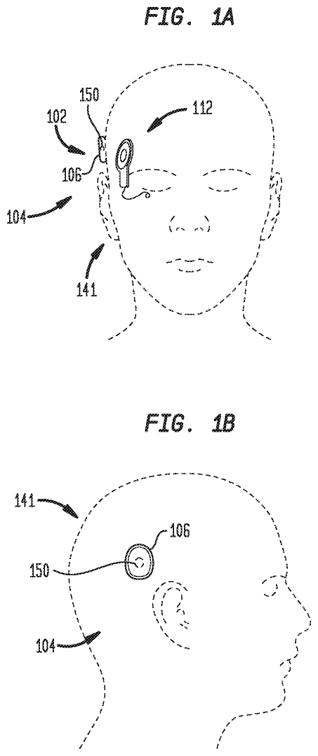

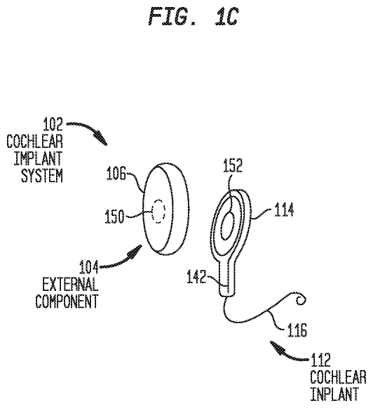

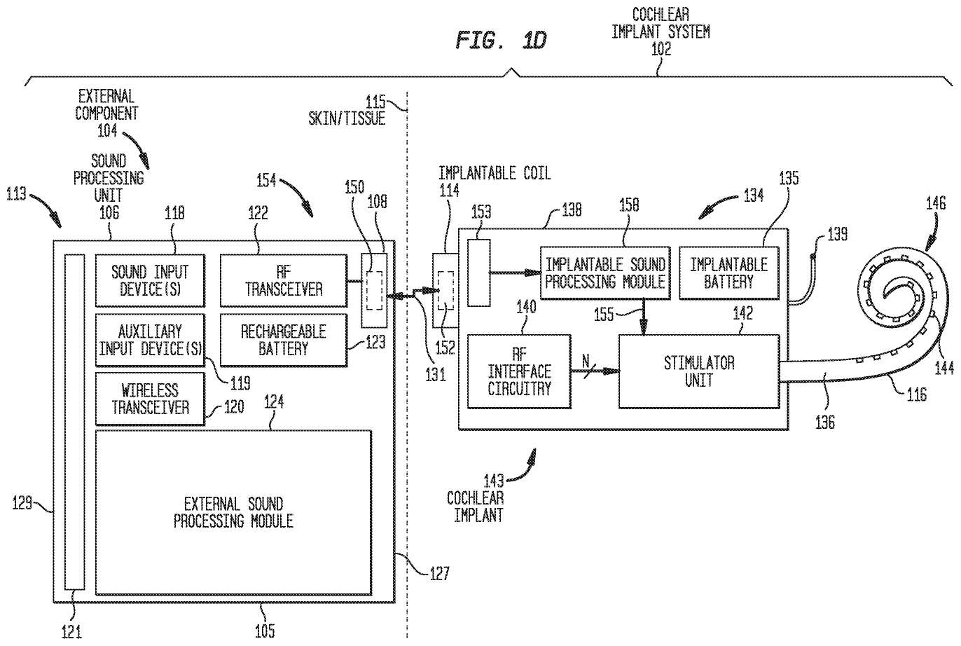

FIGS. 1A-1D illustrates an example cochlear implant system 102 configured to implement certain embodiments of the techniques presented herein. The cochlear implant system 102 comprises an external component/device 104 and an implantable component 112. In the examples of FIGS. 1A-1D, the implantable component is sometimes referred to as a “cochlear implant.” FIG. 1A illustrates the cochlear implant 112 implanted in the head 141 of a recipient, while FIG. 1B is a schematic drawing of the external device 104 worn on the head 141 of the recipient. FIG. 1C is another schematic view of the cochlear implant system 102, while FIG. 1D illustrates further details of the cochlear implant system 102. For ease of description, FIGS. 1A-1D will generally be described together.

As noted, cochlear implant system 102 includes an external device 104 that is configured to be directly or indirectly attached to the body of the recipient and an implantable component 112 configured to be implanted in the recipient. In the examples of FIGS. 1A-1D, the external device 104 comprises a sound processing unit 106, while the cochlear implant 112 includes an internal coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 105 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external device may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external devices could be located in the recipient's ear canal, worn on the body, etc.

In addition, the use of a sound processing unit as the external device, as shown in FIGS. 1A-1D, is merely illustrative. In alternative embodiments, the external device could be a device that is only configured to provide power signals to the cochlear implant 112 (e.g., a dedicated charging device) or a device that that provides power and other types of data to the cochlear implant 112.

As noted above, the cochlear implant system 102 includes the sound processing unit 106 and the cochlear implant 112. However, as described further below, the cochlear implant 112 can operate with the sound processing unit 106 stimulate the recipient or the cochlear implant 112 can operate independently from the sound processing unit 106, for at least a period, to stimulate the recipient. For example, the cochlear implant 112 can operate in a first general mode, sometimes referred to as an “external hearing” mode, in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors, or receives sound signals from another external device (e.g., via an implantable transceiver), and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

Referring first to the external hearing mode, FIGS. 1A-1D illustrate that the OTE sound processing unit 106 comprises one or more input devices 113 that are configured to receive input signals (e.g., sound or data signals). The one or more input devices 113 include one or more sound input devices 118 (e.g., one or more external microphones, audio input ports, telecoils, etc.), one or more auxiliary input devices 119 (e.g., audio ports, such as a Direct Audio Input (DAI), data ports, such as a Universal Serial Bus (USB) port, cable port, etc.), and a wireless transmitter/receiver (transceiver) 120. However, it is to be appreciated that one or more input devices 113 may include additional types of input devices and/or less input devices (e.g., the wireless short range radio transceiver 120 and/or one or more auxiliary input devices 119 could be omitted).

The OTE sound processing unit 106 also comprises the external coil 108, a charging coil 121, a closely-coupled transmitter/receiver (RF transceiver) 122, sometimes referred to as or radio-frequency (RF) transceiver 122, at least one rechargeable battery 123, and an external sound processing module 124. The external sound processing module 124 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the transceiver 140 via a hermetic feedthrough (not shown in FIG. 1D). The implant body 134 further includes an implantable rechargeable battery (implantable battery) 135.

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external device 104 to transmit data and power to the implantable component 112 via a closely-coupled wireless RF link 131 formed between the external coil 108 with the implantable coil 114. In certain examples, the closely-coupled wireless link 131 is a radio frequency (RF) link. However, various other types of energy transfer, such as infrared (IR), electromagnetic, capacitive and inductive transfer, may be used to transfer the power and/or data from an external device to an implantable component and, as such, FIG. 1D illustrates only one example arrangement.

As noted above, sound processing unit 106 includes the external sound processing module 124. The external sound processing module 124 is configured to convert received input signals (received at one or more of the input devices 113) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells and/or is configured to receives sound signals from another external device (e.g., via an implantable radio/transceiver). That is, as shown in FIG. 1D, the cochlear implant 112 can include a plurality of implantable sensors 153 configured to capture sound signals and/or can include an implantable radio (not shown in FIG. 1D) configured to receive data (sound signals) from an external device. The cochlear implant 113 also comprises an implantable sound processing module 158 that is configured to process the sound signals received via the sensors 153 and/or an implantable radio.

Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sensors 153 (or an implantable radio) are configured to detect/capture or receive signals (e.g., acoustic sound signals, vibrations, data, etc.), which are provided to the implantable sound processing module 158. The implantable sound processing module 158 is configured to convert received signals (received at one or more of the implantable sensors 153, implantable radio, etc.) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received signals into output signals 155 that are provided to the stimulator unit 142. The stimulator unit 142 is configured to utilize the output signals 155 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sensors 153 in generating stimulation signals for delivery to the recipient.

In the external hearing model, the cochlear implant 112 operates using power received from the external device 104 via the closely-coupled wireless RF link 131. That is, both power and data (e.g., processed sound signals) are transferred over the closely-coupled wireless RF link 131. The RF interface circuitry 140 is configured to provide the data to the implantable sound processing module 158 and to provide the power to the various components of the cochlear implant 112 and/or temporarily store the power. For example, the power received from the external device 104 is used to charge the implantable rechargeable battery 135, as needed.

In contrast, in the invisible hearing mode, the external device 104 is not present and/or is otherwise unable to provide power to the cochlear implant 112. As such, in the invisible hearing mode, the cochlear implant 112 is powered using power from the implantable rechargeable battery 135. The implantable rechargeable battery 135 is subsequently charged when the external device 104 is one again present and/or is otherwise again able to provide power to the cochlear implant 112.

The total amount of energy that an implantable rechargeable battery, such as implantable rechargeable battery 135, can store at any one time, often measured in terms of milliamp Hours (mAhs), is referred to herein as the “capacity” of the battery. The “cycle life” of an implantable rechargeable battery refers to the number of complete charge/discharge cycles that the battery is able to support before the capacity of the battery falls below some threshold of its original capacity so as be insufficient for its intended purpose (e.g., under 50% of the original battery capacity, under 80% of the original battery capacity, etc.) and/or the implantable rechargeable battery has a sufficiently high charge-time to run-time ratio so as be insufficient for its intended purpose. For example, upon initial operation, an implantable rechargeable battery may need two (2) hours to be charged to a level that provides a capacity for a sixteen (16) hour run time of the associated implantable component (e.g., a charge-time to run-time ratio of 0.125). However, as the battery is cycled over time (e.g., repeatedly discharged and charged), the charge time may drop to 1 hour, but the resulting usage time would drop more significantly (e.g., the capacity of the battery decreases) to 2 hours (e.g., a charge-time to run-time ratio of 0.5). A charge-time to run-time ratio above a predetermined threshold means that too much charging time is required, for the resulting run-time, such that the rechargeable battery is not operating as intended (e.g., to power the implantable component for sufficiently long periods of time without being charged).

An implantable rechargeable battery that has reached the end of its cycle life, as noted above, is sometimes referred to herein as a “dead” or an “end-of-life (EOL)” rechargeable battery. A rechargeable battery can also prematurely reach its end-of-life when the battery experiences an unexpected failure or fault (e.g., short circuit, inadvertent charge, lithium plating swell/leak, etc.), such that the battery can no longer be safely charged and/or can no longer be safely used to power an implantable component.

All rechargeable batteries have a limited lifespan and they will eventually reach their end-of-life as a result of ongoing cycling. In general, higher capacity batteries have shorter cycle lives than lower capacity batteries. The “calendar life” of a rechargeable battery refers to the time period (e.g., in terms of years) when the rechargeable battery is expected to reach its end-of-life.

Cochlear implants and other implantable medical devices are increasingly progressing towards the use of integrated implantable rechargeable batteries (e.g., rechargeable monolithic battery cells) where the implantable rechargeable batteries cannot be replaced. For example, in many conventional arrangements, due to the device design, location of the implantable battery, etc., when an implantable rechargeable battery is end-of-life, the entire implantable component (including the battery) needs to be explanted (removed from the recipient) and replaced. That is, if an end-of-life rechargeable battery is integrated within an implantable component implant and is completely non-replaceable, then at least the component that includes the end-of-life battery needs to be replaced. At a minimum, even if an end-of-life rechargeable battery can be replaced without replacing the entire implantable component, a surgical procedure is still required to remove the end-of-life rechargeable battery, implant a new rechargeable battery, connect the new battery, etc.

Implantable rechargeable batteries, while extremely safe, can fail or age prematurely (e.g., short circuit, inadvertent charge, experience lithium plating swell/leak, etc.). Additionally, as noted, implantable rechargeable batteries have a limited lifespan and will eventually reach their end-of-life as a result of ongoing cycling. In conventional arrangements, there is no mechanism by which the health of an implantable rechargeable battery can be monitored to ensure that the battery (and the system) remains safe once the battery reaches its end-of-life. Conventional systems also lack the ability to permanently disconnect an end-of-life battery. Accordingly, in conventional arrangements, when the rechargeable battery reach its end-of-life (e.g., where the battery retains no or little charge, experiences a fault that prevents continued use of the battery, the implantable battery and, potentially the entire implantable component, needs to be explanted. Furthermore, existing systems do not provide a mechanism enabling a recipient to extend the calendar life of an implantable rechargeable battery.

However, certain medical device systems, such as certain cochlear implant systems, are configured to be powered by implantable rechargeable battery or by an external device. The techniques presented herein leverage the ability to receive power from an external device so as to provide the ability to continue use of the implantable component, even when the implantable rechargeable battery is end-of-life. That is, the techniques presented herein provide a mechanism to temporarily or permanently disconnect an implantable rechargeable battery while allowing the associated implantable component to continue to operate safely with the implantable rechargeable battery disconnected.

More specifically, the techniques presented herein provide a recipient with battery capacity and battery life (e.g., cycle life, calendar life, etc.) information and the ability to temporarily disconnect the implantable rechargeable battery to extend the calendar life of the implantable rechargeable battery (e.g., extend the time until the reaches its end-of-life as a result of cycling). Additionally, the techniques presented provide the ability to permanently disconnect an implantable rechargeable battery once it reaches its end-of-life so that the implantable component can continue operation using only power received from an external device, as well as the ability to continue to monitor the permanently disconnected implantable rechargeable battery.

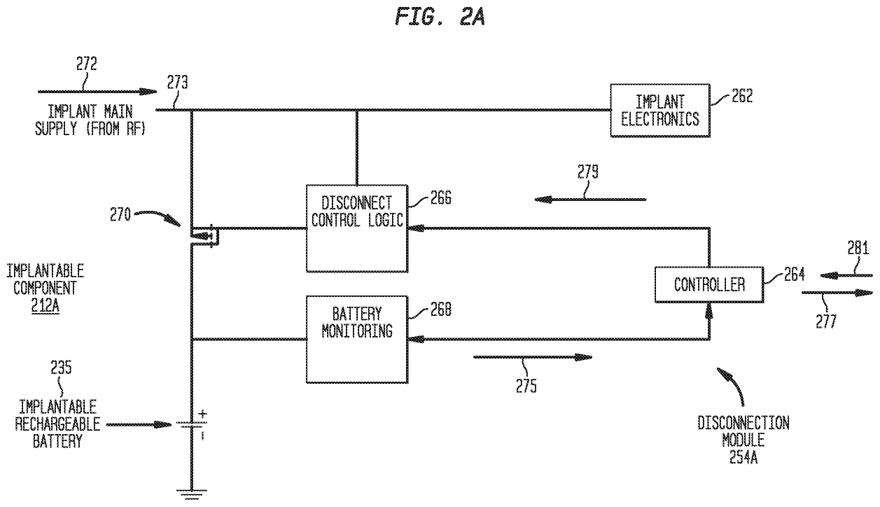

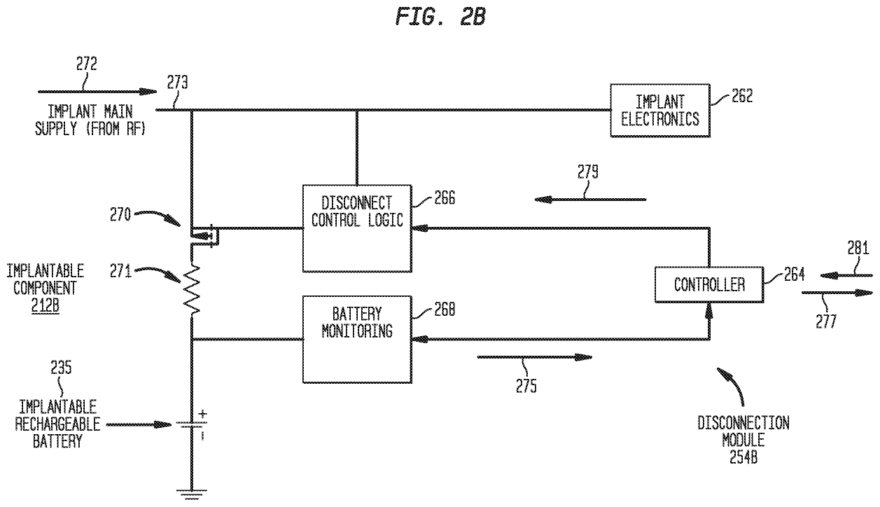

FIG. 2A is a functional block diagram illustrating elements of an implantable component, such as a cochlear implant, in accordance with embodiments presented herein. Merely for ease of description, the implantable component of FIG. 2A is referred to as implantable component 212A.

As shown in FIG. 2A, the implantable component 212A comprises an implantable rechargeable battery (implantable battery) 235, implant electronics 262 representing the various operational electrical components of the implantable component 212A, and a battery disconnection module 254A. The battery disconnection module 254A comprises a controller 264, disconnect control logic 266, a battery monitoring module 268, and a switch module 270. The various modules/logic 262, 264, 266, and 268 can be implemented by any of hardware, software, and/or combinations thereof. In the context of a cochlear implant, the implant electronics include, for example, an implantable sound processing module, a stimulator unit, etc.

As shown in FIG. 2A, external power signals 272 are received at a node 273. In the example of FIG. 2A, the external power signals 272 are received from an external device via an implantable inductive coil (not shown in FIG. 2A) and a tank circuit (also not shown in FIG. 2A). As such, node 273 represents the output of an implantable tank circuit that includes an implantable inductive coil. In normal operation, the external power signals 272 are used to charge the implantable rechargeable battery 235 and, when present, the external power signals 272 are the main source of power for the implant electronics 262. That is, when the external power signals 272 are present (being received from the external device), the implant electronics 262 are powered using the external power signals 272, and the implantable rechargeable battery 235 is not discharged. However, when the external power signals 272 are not present, the implant electronics 262 are powered using energy stored in, and provided by, the implantable charge battery 235.

As noted above, due to the presence of implantable charge battery 235, implantable component 212A can operate, for periods of time, without the need to receive power from an external device. During these periods of time, the implantable charge battery 235 will be discharged and subsequently charged when external power signals 272 are again received from an external device. Therefore, over time, implantable rechargeable battery 235 will be cycled (discharged and charged) repeatedly and will eventually reach its end-of-life (EOL) as a result of the ongoing cycling (e.g., reach a state where there battery capacity reduces to a point where they no longer hold sufficient charge for the recipient).

In the example of FIG. 2A, the battery disconnection module 254A (e.g., controller 264, disconnect control logic 266, battery monitoring module 268, and switch module 270) represents the components (e.g., circuitry) that can both connect and disconnect the implantable rechargeable battery 235, as well as monitor a health/state of the implantable rechargeable battery 235. It is to be appreciated that the specific separation of functions/modules, as shown in FIG. 2A, is merely illustrative and does not imply or require any specific hardware or software implementation and that the techniques presented herein can be implemented by a number of different structural and functional arrangements. For example, as noted elsewhere herein, the controller 264, disconnect control logic 266, battery monitoring module 268, and switch module 270 could be implemented by one or more integrated circuits, such as an application specific integrated circuit (ASIC), a combination of hardware/software elements, etc. It is also to be appreciated that the battery disconnection module 254A may, at least in part, form part of the implant electronics 262. For example, the controller 264 and battery monitoring module 268 could be implemented by the same one or more processors that perform sound processing operations in a cochlear implant or other auditory prosthesis.

Returning to the example of FIG. 2A, the battery monitoring module 268 is configured to monitor the state/health of the implantable battery 235 and provide battery health information 275 to the controller 264. It is to be appreciated that the monitoring of the health of the implantable rechargeable battery 235 could involve a number of aspects and could be implemented in a number of different manners. For example, the battery monitoring module 268 can be configured to monitor the voltage of the implantable rechargeable battery 235 (e.g., when the battery is connected or disconnected) and confirm the voltage is within acceptable limits (e.g., above a minimum threshold voltage and/or below a maximum threshold voltage). The battery voltage could be monitored using, for example, an analog-to-digital (A/D) converter to measure battery voltage. Alternatively, the battery monitoring module 268 can be configured to monitor the current within the battery. In the context of a disconnected battery, the battery monitoring module 268 could charge the disconnected battery with a small current and measure the battery voltage in response to this charge current (e.g., to check that the battery is not leaking). It is to be appreciated that these battery monitoring techniques are merely illustrative and that the battery monitoring module 268 could monitor the health of the implantable battery 235 in other manners.

The controller 264 is configured to receive and analyze the battery health information 275 and, potentially, initiate one or more operations based thereon. For example, the controller 264 can be configured to provide battery status data 277 (e.g., the battery health information 275 or other battery information generated from the battery health information 275) to the recipient or other user. The battery status data 277 can be provided to the recipient or other user in a number of different manners. For example, the battery status data 277 could be provided to the recipient in an audible format (e.g., via a signal delivered to recipient via the implantable component 212, via audible signals generated by an external device, etc.) and/or in a visual format (e.g., via a display screen of an external device). In general, the battery status data 277 can comprise information about the state/health of the battery 235, information relating to remaining life of the battery 235. In certain embodiments, the battery status data 277 can include an estimate of the remaining cycle life of the battery, an estimate of the calendar life of the battery, an indication of whether or not the implantable battery 235 is connected or disconnected, an indication of any abnormal behavior or characteristics of the battery that requires the recipient to attend a clinic for assessment, changes in battery parameters (e.g., battery impedance), changes in rate of battery voltage increase or decrease, etc.

FIG. 2A illustrates an example in which the battery status data 277 is generated by the controller 264. It is be appreciated that, in alternative embodiments, the battery status data 277 could alternatively be generated at an external device, such as an external component, mobile phone, etc., in wireless communication with the implantable component 212A. For example, the controller 264 could be configured to wireless send the battery health information 275 to an external device (e.g., directly via a wireless interface within the implantable component 212A or indirectly via the implantable inductive coil and an external device). In such embodiments, the external device can be configured to analyze the battery health information 275, generate the battery status data 277, and provide the battery status data 277 to a user (e.g., via a display screen of the external device).

In addition, the controller 264 interfaces with the disconnect control logic 266 to control the configuration of switch module 270. In one example, the disconnect control logic 266 is a feedback control loop that actively controls the state of the switch module 270 based on control signals 279 from the controller 264. In this way, the disconnect control logic 266 and controller 264 can selectively “connect” or “disconnect” the implantable rechargeable battery 235. As noted above, implantable rechargeable battery 235 is “connected” when the battery is capable of being charged by power signals 272 received from an external device and is able to supply power to the implant electronics 262. In contrast, implantable rechargeable battery 235 is “disconnected” when the battery is incapable of being charged by power signals 272 received from an external device and, in certain embodiments, is incapable of supplying power to the implant electronics 262.

In the example of FIG. 2A, when the implantable rechargeable battery 235 is disconnected, the implantable component 212A is forced to operate using the power signals 272. That is, when the implantable rechargeable battery 235 is disconnected, the implantable component 212A can only operate when an external device is present and supplying the power signals 272.

The connection and/or disconnection of the implantable rechargeable battery 235 may be implemented in a number of different manners. For example, in certain embodiments, the switch module 270 may comprise one or more switches (e.g., an arrangement of MOSFETS) connected between node 273 and the implantable rechargeable battery 235 to physically connect/disconnect (e.g., switch in and out) the implantable rechargeable battery 235 from node 273 and the implant electronics 262. That is, the one or more switches can be closed or opened to form a physical disconnection/break between the implantable rechargeable battery 235 and node 273 and/or the implant electronics 262.

In alternative embodiments, shown in FIG. 2B, disconnection of the implantable rechargeable battery 235 does not involve creation of a physical disconnection/break in the circuit between the implantable rechargeable battery 235 and node 273 or the implant electronics 262. For example, shown in FIG. 2B is an implantable component 212B that is similar to the implantable component 212A of FIG. 2A except that disconnection of the implantable rechargeable battery 235 can include connection of one or more high impedance elements 271 (e.g., one or more resistors) between the implantable rechargeable battery 235 and node 273 or the implant electronics module 262 to prevent or limit the flow of current to the implantable between the implantable rechargeable battery 235 and node 273 to limit the battery current to a level at which the battery is not charged. That is, in FIG. 2B, the battery disconnection module 254B includes one or more additional high impedance elements 271 that are placed in series with the switch 270 to prevent or limit the flow of current to the implantable rechargeable battery 235.

The above examples of FIGS. 2A and 2B are merely illustrative of techniques that can be used to disconnect the implantable rechargeable battery 235. As such, it is be appreciated that, in accordance with other embodiments presented herein, the switch module 270 could operate in other manners to selectively disconnect the implantable rechargeable battery 235 (e.g., prevent charging of the implantable rechargeable battery 235).

Additionally, FIGS. 2A and 2B illustrate example arrangements in which the switch module 270 disconnects/connects the implantable rechargeable battery 235 from both node 273 (e.g., prevents the implantable rechargeable battery 235 from being charged) and from implant electronics 262 (e.g., prevents implantable rechargeable battery 235 from supplying power to the implant electronics 262). It is to be appreciated that these arrangements are merely illustrative and that, in alternative embodiments, different/separate components may be used to prevent the implantable rechargeable battery 235 from being charged and to prevent the implantable rechargeable battery 235 from supplying power to the implant electronics 262.

As noted above, in the example of FIGS. 2A and 2B, the controller 264 interfaces with the disconnect control logic 266 to control the configuration of switch module 270 and, accordingly, control connection or disconnection of the implantable battery 235 (e.g., from node 273 and/or from the implant electronics 262). In the accordance with certain examples, the controller 264 is configured to temporarily (e.g., reversibly) disconnect the implantable battery 235. In accordance with the same or other examples, the controller 264 is configured to permanently (i.e., non-reversibly) disconnect the implantable battery 235 from the implant electronics 262.

The controller 264 can be configured to temporarily disconnect the implantable battery 235 in response to control signals received from an external device (e.g., an external device, mobile device, etc.). For example, as the implantable battery 235 ages due to cycling, the battery will retain less and less capacity and, eventually, approach its end-of-life. However, the recipient of the implantable component 212 may wish to be able to extend the calendar life of the implantable battery 235 by, at certain times, proactively limiting the cycling of the implantable rechargeable battery 235 while maintaining the implantable rechargeable battery 235 in a state that provide the option to continue using the implantable rechargeable battery 235 in the future, if desired. For example, a recipient may choose to only use the implantable battery 235 for “special occasions” where they want to avail of the discreteness of operation without an external device (e.g., invisible hearing in the context of an auditory prosthesis), while at other times require/force use the external device (e.g., force use of “external hearing” at other times). In such embodiments, the recipient can use an external device to send a control signal 281 to the controller 264 (e.g., directly via a wireless interface within the implantable component 212 or indirectly via the implantable inductive coil and an external device) that causes the controller 264 to operate switch module 270 to temporarily disconnect the implantable battery 235 from the implant electronics 262. At a subsequent time, the recipient can again use the external device to send another control signal 281 to the controller 264 that causes the controller 264 to operate switch module 270 to connect the implantable battery 235.

As noted, by temporarily disconnecting the implantable battery, the implantable battery 235 is not cycled (e.g., not discharged and charged) and the implantable component 212 can only operate using power received from an external device. That is, while the implantable battery 235 is connected, the implantable components 212A or 212B will only operate when power signals 272 are received from the external device. By limiting operation to use of the power signals 272, the implantable battery 235 is not cycled and it is possible to extend the calendar life of the battery (e.g., by months to several years). The implantable rechargeable battery 235 can again be reconnected to the system if the recipient again wants use of the implantable battery (e.g., wants to use invisible hearing in the future). Accordingly, with an auditory prosthesis, the techniques presented herein preserve the facility of invisible hearing for longer without revision surgery.

As noted, the selective temporary disconnection of the implantable battery 235 can extend the calendar life of the battery. However, as some point in time, the implantable battery 235 will reach its end-of-life (e.g., true end-of-life as a result of cycling, experience a fault such that battery can only be used to power the implantable component, etc.). Once the implantable battery 235 reaches its end-of-life, the controller 264 is configured to permanently disconnect the implantable battery 235. In accordance certain examples presented herein, the controller 264 is configured to automatically permanently disconnect the implantable battery 235 based on battery health information 275. For example, as noted above, the controller 264 can be configured to analyze the battery health information 275, determine the implantable rechargeable battery 235 has reached its end-of-life, and accordingly operate switch module 270 to disconnect the implantable battery 235.

The controller 264, or an external device, can determine that the implantable rechargeable battery 235 has reached its end-of-life in a number of different manners. In certain embodiments, the controller 264 or an external device determines that the implantable rechargeable battery 235 has reached its end-of-life by determining that a cycle-life of the implantable rechargeable battery 235 is below a selected/predetermined threshold. In further embodiments, the controller 264 or an external device determines that the implantable rechargeable battery 235 has reached its end-of-life by determining that the implantable rechargeable battery 235 has experienced a fault condition making continued operation of the implantable rechargeable battery 235 unsafe for the recipient. In other embodiments, the controller 264 or an external device determines that the implantable rechargeable battery 235 has reached its end-of-life by determining that a capacity of the implantable rechargeable battery 235 is below a predetermined threshold. In still other embodiments, the controller 264 or an external device determines that the implantable rechargeable battery 235 has reached its end-of-life by determining that a charge-time to run-time ratio of the implantable rechargeable battery 235 is above a predetermined threshold.

In alternative embodiments, the controller can be configured to permanently disconnect the implantable battery 235 in response to a control signal received 281 from an external device (e.g., an external device, mobile device, etc.). In such embodiments, the recipient can use an external device to send a control signal 281 to the controller 264 that causes the controller 264 to operate switch module 270 to permanently disconnect the implantable battery 235. As noted, a permanent disconnection of implantable battery 235 means that the implantable rechargeable battery 235 can no longer be charged, but that the implantable component 212 can continue operation using external power signals 272. Accordingly, implementation of the techniques presented herein means that explant of the implantable component 212 or the implantable rechargeable battery 235 is not mandatory when the implantable rechargeable battery 235 reaches its end-of-life. While disconnected, the battery monitoring module 268 is configured to continue to monitor the health of the battery (e.g., to provide assurance to the recipient or other user that the implantable component 212A or 212B remains safe to use).

In summary, FIGS. 2A and 2B illustrate arrangements for ongoing health monitoring of implantable battery 235, while allowing the implantable components 212A and 212B to continue to operate with a temporarily or permanently disconnected battery. That is, FIGS. 2A and 2B generally illustrate arrangements to selectively disconnect the implantable battery 235 (e.g., a circuit to prevent charge and discharge of the implant battery when an external power source is available), but still enable the implantable component to safely continue operation even with the battery disconnected. The arrangement of FIGS. 2A and 2B also monitor the health of the implantable battery 235 so that battery is maintained in a state that preserves longevity.

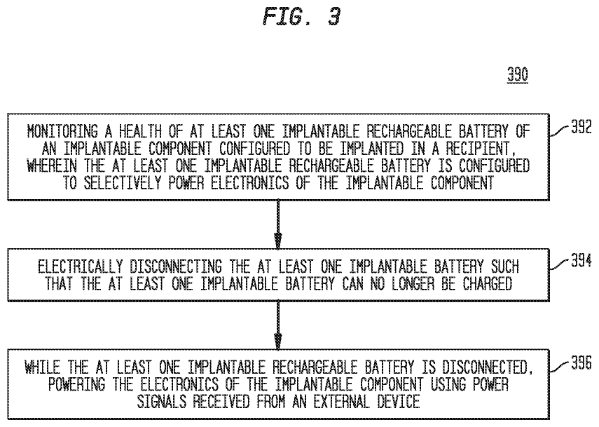

FIG. 3 is a flowchart of an example method 390, in accordance with certain embodiments presented. Method 390 begins at 392 where a health of at least one implantable rechargeable battery of an implantable component configured to be implanted in a recipient is monitored. The at least one implantable rechargeable battery is configured to selectively power electronics of the implantable component. At 394, the at least one implantable battery is electrically disconnected such that the at least one implantable battery can no longer be charged. At 396, while the at least one implantable rechargeable battery is disconnected, the electronics of the implantable component are powered using power signals received from an external device.

As noted elsewhere herein, embodiments presented herein have been primarily described with reference to an example auditory prosthesis system, namely a cochlear implant system. However, as noted above, it is to be appreciated that the techniques presented herein may be implemented by a variety of other types of implantable medical devices (or systems that include other types of implantable medical devices). For example, the techniques presented herein may be implemented by other auditory prostheses, such as acoustic hearing aids, middle ear auditory prostheses, bone conduction devices, direct acoustic stimulators, electro-acoustic prostheses, other electrically simulating auditory prostheses (e.g., auditory brain stimulators), etc. The techniques presented herein may also be implemented by tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

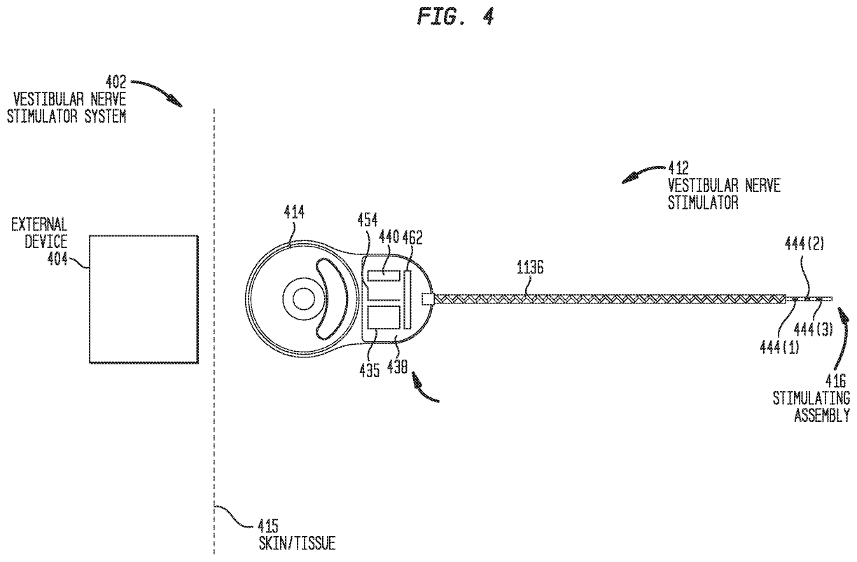

For example, FIG. 4 illustrates an example vestibular stimulator system 402 in accordance with embodiments presented herein. In this example, the vestibular stimulator system 402 comprises an implantable component (vestibular stimulator) 412 and an external device/component 404 (e.g., external processing device, battery charger, remote control, etc.).

The vestibular stimulator 412 comprises an implant body (main module) 434, a lead region 436, and a stimulating assembly 416, all configured to be implanted under a skin/tissue flap (skin flap) 415 of the recipient. The implant body 434 generally comprises a hermetically-sealed housing 438 in which RF interface circuitry 440, one or more rechargeable batteries 435, implant electronics 462, and a battery disconnection module 454 are disposed. The implant body 434 also includes an internal/implantable coil 414 that is generally external to the housing 438, but which is connected to the RF interface circuitry 440 via a hermetic feedthrough (not shown).

The vestibular stimulator 412 also comprises stimulating assembly 416, which includes a plurality of electrodes 444 disposed in a carrier member (e.g., a flexible silicone body). In this specific example, the stimulating assembly 416 comprises three (3) stimulation electrodes, referred to as stimulation electrodes 444(1), 444(2), and 444(3). The stimulation electrodes 444(1), 444(2), and 444(3) function as an electrical interface for delivery of electrical stimulation signals to the recipient's vestibular system.

The stimulating assembly 416 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In accordance with embodiments presented herein, the external device 404 can include one or more batteries 423, an RF transceiver 422, and an external coil 408 that is configured to be wirelessly (e.g., inductively) coupled to the implantable coil 414 of the vestibular stimulator 412. After the vestibular stimulator 412 is implanted within the recipient's head, the external device 404 can attached to the head of the recipient and can provide power, and potentially data, to the vestibular stimulator 412. That is, the vestibular stimulator 412 can operate using power signals received from the external device 404.

As noted above, the vestibular stimulator 412 also comprises one or more implantable rechargeable batteries 435. Due to the presence of the one or more implantable rechargeable batteries 435, vestibular stimulator 412 can operate, for periods of time, without the need to receive power from the external device 404. During these periods of time, the one or more implantable rechargeable batteries 435 will be discharged and subsequently charged when external power signals are again received from the external device 404. Therefore, over time, implantable one or more implantable rechargeable batteries 435 will be cycled (discharged and charged) repeatedly and will eventually reach their end-of-life (EOL) as a result of the ongoing cycling (e.g., reach a state where there battery capacity reduces to a point where they no longer hold sufficient charge for the recipient).

In the example of FIG. 4, the battery disconnection module 454, which may be implemented similar to battery disconnection module 254A of FIG. 2A, is configured is configured to monitor the state/health of the one or more implantable rechargeable batteries 435 and selectively disconnect the one or more implantable rechargeable batteries 435 from the implant electronics 462.

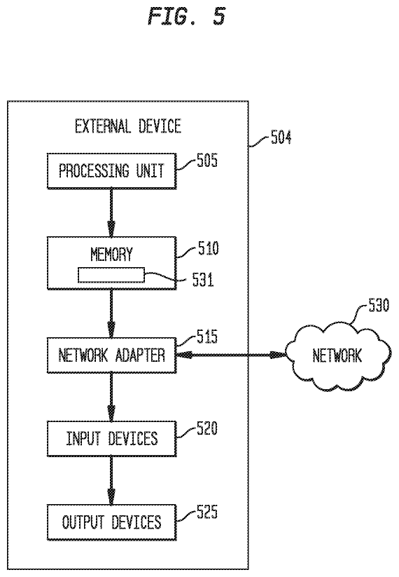

As noted above, aspects of the techniques presented herein can be implemented at an externa device in wireless communication with an implantable component. FIG. 5 illustrates an example of a suitable external device 504 with which one or more of the disclosed examples can be implemented. Computing systems, environments, or configurations that can be suitable for use with examples described herein include, but are not limited to, personal computers, server computers, hand-held devices, laptop devices, multiprocessor systems, microprocessor-based systems, programmable consumer electronics (e.g., smart phones), network PCs, minicomputers, mainframe computers, tablets, distributed computing environments that include any of the above systems or devices, and the like. The external device 504 can be a single virtual or physical device operating in a networked environment over communication links to one or more remote devices. The remote device can be an auditory prosthesis (e.g., an auditory prosthesis), a personal computer, a server, a router, a network personal computer, a peer device or other common network node.

In its most basic configuration, external device 504 includes at least one processing unit 505 and memory 510. The processing unit 505 includes one or more hardware or software processors (e.g., Central Processing Units) that can obtain and execute instructions. The processing unit 505 can communicate with and control the performance of other components of the external device 504.

The memory 510 is one or more software or hardware-based computer-readable storage media operable to store information accessible by the processing unit 505. The memory 510 can store, among other things, instructions executable by the processing unit 505 to implement applications or cause performance of operations described herein, as well as other data. The memory 510 can be volatile memory (e.g., RAM), non-volatile memory (e.g., ROM), or combinations thereof. The memory 510 can include transitory memory or non-transitory memory. The memory 510 can also include one or more removable or non-removable storage devices. In examples, the memory 510 can include RAM, ROM, EEPROM (Electronically-Erasable Programmable Read-Only Memory), flash memory, optical disc storage, magnetic storage, solid state storage, or any other memory media usable to store information for later access. In examples, the memory 510 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 510 can include wired media such as a wired network or direct-wired connection, and wireless media such as acoustic, RF, infrared and other wireless media or combinations thereof. In certain embodiments, the memory 510 comprises battery control logic 535 that, when executed, enables the processing unit 505 to perform aspects of the techniques presented.

In the illustrated example, the external device 504 further includes a network adapter 515, one or more input devices 520, and one or more output devices 525. The system 504 can include other components, such as a system bus, component interfaces, a graphics system, a power source (e.g., a battery), among other components.

The network adapter 515 is a component of the external device 504 that provides network access (e.g., access to at least one network 530). The network adapter 515 can provide wired or wireless network access and can support one or more of a variety of communication technologies and protocols, such as ETHERNET, cellular, BLUETOOTH, near-field communication, and RF (Radiofrequency), among others. The network adapter 515 can include one or more antennas and associated components configured for wireless communication according to one or more wireless communication technologies and protocols.

The one or more input devices 520 are devices over which the external device 504 receives input from a user. The one or more input devices 520 can include physically-actuatable user-interface elements (e.g., buttons, switches, or dials), touch screens, keyboards, mice, pens, and voice input devices, among others input devices.

The one or more output devices 525 are devices by which the external device 504 is able to provide output to a user. The output devices 525 can include, displays, speakers, and printers, among other output devices.

It is to be appreciated that the arrangement for external device 504 shown in FIG. 5 is merely illustrative and that aspects of the techniques presented herein may be implemented at a number of different types of systems/devices. For example, the external device 504 could be a laptop computer, tablet computer, mobile phone, surgical system, etc.

It is to be appreciated that the embodiments presented herein are not mutually exclusive and that the various embodiments may be combined with another in any of a number of different manners.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A method, comprising:
monitoring a health of at least one implantable rechargeable battery of an implantable component configured to be implanted in a recipient, wherein the at least one implantable rechargeable battery is configured to selectively power electronics of the implantable component;
electrically disconnecting the at least one implantable rechargeable battery such that the at least one implantable rechargeable battery can no longer be charged; and
while the at least one implantable rechargeable battery is disconnected, powering the electronics of the implantable component using power signals received from an external device.

2. The method of claim 1, wherein monitoring the health of the at least one implantable rechargeable battery comprises:
obtaining battery health information from the at least one implantable rechargeable battery; and
determining, based on the battery health information, that the at least one implantable rechargeable battery has reached an end-of-life.

3. The method of claim 2, wherein that the at least one implantable rechargeable battery has reached an end-of-life comprises:
determining that a cycle-life of the at least one implantable rechargeable battery is below a selected threshold.

4. The method of claim 2, wherein determining that the at least one implantable rechargeable battery has reached an end-of-life comprises:
determining that the at least one implantable rechargeable battery has experienced a fault condition making continued operation of the at least one implantable rechargeable battery unsafe for the recipient.

5. The method of claim 1, wherein determining that the at least one implantable rechargeable battery has reached an end-of-life comprises:
determining that a capacity of the at least one implantable rechargeable battery is below a predetermined threshold.

6. The method of claim 1, wherein determining that the at least one implantable rechargeable battery has reached an end-of-life comprises:
determining that a charge-time to run-time ratio of the at least one implantable rechargeable battery is above a predetermined threshold.

7. The method of claim 1, wherein disconnecting the at least one implantable rechargeable battery from the electronics comprises:
permanently disconnecting the at least one implantable rechargeable battery from the electronics.

8. The method of claim 1 wherein disconnecting the at least one implantable rechargeable battery comprises:
temporarily disconnecting the at least one implantable rechargeable battery.

9. The method of claim 8, further comprising:
reconnecting the at least one implantable rechargeable battery such that at least one implantable rechargeable battery is able to provide power to the electronics.

10. The method of claim 1, wherein disconnecting the at least one implantable rechargeable battery comprises:
disconnecting the at least one implantable rechargeable battery in response to receipt of a control signal from an external device.

11. The method of claim 1, wherein disconnecting the at least one implantable rechargeable battery comprises:
forming a physical disconnection between the at least one implantable rechargeable battery and a circuit node at which the power signals are received from the external device.

12. The method of claim 11, wherein disconnecting the at least one implantable rechargeable battery further comprises:
forming a physical disconnection between the at least one implantable rechargeable battery and the electronics.

13. The method of claim 1, wherein disconnecting the at least one implantable rechargeable battery comprises:
placing one or more high impedance circuit components between the at least one implantable rechargeable battery and a circuit node that which the power signals are received from the external device.

14. The method of claim 1, further comprising:
while implantable rechargeable battery is disconnected, continuing to monitor a health of the at least one implantable rechargeable battery.

15. An implantable component configured to be implanted in a recipient, comprising:
an inductive coil configured to receive power signals from an external device;
at least one implantable rechargeable battery configured to be charged using power signals received at the inductive coil from the external device;
implant electronics configured to be powered by power received from the at least one implantable rechargeable battery; and
a battery disconnection module configured to selectively disconnect the at least one implantable rechargeable battery from the implant electronics and the inductive coil,
wherein when the at least one implantable rechargeable battery is disconnected, the implant electronics are only powered using power signals received from the external device.

16. The implantable component of claim 15, wherein the battery disconnection module is configured to:
selectively disconnect the at least one implantable rechargeable battery such that the at least one implantable rechargeable battery is unable to receive a current from the inductive coil that is sufficient to charge the at least one implantable rechargeable battery.

17. The implantable component of claim 15, wherein the battery disconnection module comprises one or more switch modules configured to form a physical disconnection between the at least one implantable rechargeable battery and the inductive coil and between the least one implantable rechargeable battery the implant electronics.

18. The implantable component of claim 17, wherein the one or more switch modules are configured to place one or more high impedance circuit components between the at least one implantable rechargeable battery and the inductive coil.

19. The implantable component of claim 15, wherein the battery disconnection module is configured to:
obtain battery health information from the at least one implantable rechargeable battery; and
monitor a health of the at least one implantable rechargeable battery based on the battery health information.

20. The implantable component of claim 19, wherein the battery disconnection module is configured to:
automatically disconnect the at least one implantable rechargeable battery from the inductive coil and the implant electronics based on the health of the at least one implantable rechargeable battery.

21. The implantable component of claim 19, wherein the battery disconnection module is configured to:
wirelessly send battery status data indicating the health of the at least one implantable rechargeable battery to an external computing device.

22. The implantable component of claim 19, wherein the battery disconnection module comprises a controller configured to:
determine, based on the battery health information, that the at least one implantable rechargeable battery has reached an end-of-life; and
permanently disconnect the at least one implantable rechargeable battery from the inductive coil and implant electronics.

23. The implantable component of claim 22, wherein the controller is configured to determine that the at least one implantable rechargeable battery has reached an end-of-life when a cycle-life of the at least one implantable rechargeable battery is below a selected threshold.

24. The implantable component of claim 22, wherein the controller is configured to determine that the at least one implantable rechargeable battery has reached an end-of-life when the at least one implantable rechargeable battery has experienced a fault condition making continued operation of the at least one implantable rechargeable battery unsafe for the recipient.

25. The implantable component of claim 22, wherein the controller is configured to determine that the at least one implantable rechargeable battery has reached an end-of-life when a capacity of the at least one implantable rechargeable battery is below a predetermined threshold.

26. The implantable component of claim 22, wherein the controller is configured to determine that the at least one implantable rechargeable battery has reached an end-of-life when a charge-time to run-time ratio of the least one implantable rechargeable battery is above a predetermined threshold.

27. The implantable component of claim 22, wherein the controller is configured to permanently disconnect the at least one implantable rechargeable battery only in response to receipt of a control signal from an external computing device.

28. (canceled)

29. A method, comprising:
generating battery status data associated with at least one implantable rechargeable battery of an implantable component configured to be implanted in a recipient;
sending the battery status data to an external computing device;
receiving one or more control signals from the external computing device indicating that the at least one implantable rechargeable battery should be disconnected; and
in response to receipt of the one or more control signals, electrically disconnecting the at least one implantable rechargeable battery such that the at least one implantable rechargeable battery can no longer be charged.

30. (canceled)

31. The method of claim 29, wherein the at least one implantable rechargeable battery is temporarily disconnected, and wherein the method further comprises:
receiving at least one control signal from the external computing device indicating that the at least one implantable rechargeable battery should be reconnected; and
in response to receipt of the at least one control signal, electrically reconnecting the at least one implantable rechargeable battery such that the at least one implantable rechargeable battery can be charged.

32. The method of claim 29, wherein generating battery status data associated with at least one implantable rechargeable battery comprises:
obtaining battery health information from the at least one implantable rechargeable battery; and
determining, based on the battery health information, that the at least one implantable rechargeable battery has reached an end-of-life.

33-44. (canceled)
